(19) United States
(12) Patent Application Publication
(10) Pub. No.: US 2006/0199191 A1
(43) Pub. Date: Sep. 7, 2006

(54) Methods and compositions for the synthesis of RNA and DNA

(76) Inventors: Demian Obregon, St. Petersburg, FL (US); Mussa Ali, St. Petersburg, FL (US)

Correspondence Address: Demian Obregon, 501 37th Ave. North East, St. Petersburg, FL, 33704, US

(21) Appl. No.: 11/164,961

(22) Filed: Dec. 12, 2005

Related U.S. Application Data
(60) Provisional application No. 60/593,122, filed on Dec. 13, 2004.

(51) Int. Cl. C12Q 1/68; C12P 19/34

(52) U.S. Cl. 435/6; 435/91.2

(57) ABSTRACT

Methods for the production of duplexes and single-stranded RNA and/or DNA of a desired length and sequence based on a novel template design which incorporates 2 polymerase promoters, primers, and production sequences within a single molecule are provided. This single molecule template design allows high-efficiency, high-yield production of single or multiple nucleic acid molecules in a single reaction vessel and thus is amenable to high-throughput automation. This single molecule design also allows easy incorporation of single molecule templates into delivery vectors for either in vitro, ex vivo, in vivo, or therapeutic application. Methods for producing single template molecule-based RNA or DNA molecules, or hybrid molecules, in vivo and therapeutic uses for such molecules are provided. Single molecule template kit designs are also described.

### BACKGROUND OF THE INVENSION

1. Field of Invention

Inventions related to methods and compositions for synthesizing DNA, RNA and DNA/RNA hybrids, duplexes and single-stranded RNA and/or DNA of a desired length and sequence based on the use of a single opposed template molecule, in vitro, ex vivo, and in vivo.

2. Description of the Related Art

Simple and efficient methods for the generation of RNA and DNA have long been sought after in the fields of molecular biology, biotechnology and genetic engineering. Although major advances have been made in efforts to effect efficient RNA and DNA generation, more efficient methods were still needed.

Prior techniques used to concurrently generate multiple strands RNA and DNA have been rendered inefficient either by their inability to synthesize multiple nucleic-acid strands concurrently or by their necessity to implement multiple steps in order to synthesize multiple strands. Other techniques use large circular forms of nucleic acids such as plasmids, or cosmids to produce RNA or DNA. These techniques necessitate the inclusion of steps to splice in a desired production sequence, using endonucleases, in order to produce the desired nucleic acid thus adding more processing to the overall production of RNA or DNA. Similarly the use of viral constructs require the use of endonucleases to splice in the production sequence(s) of choice in order to produce the resultant nucleic acid strand of choice.

Recently, advances in nucleic acid production have spawned new approaches. These approaches are described below.

Single production sequence, single promoter sequence approach to nucleic acid strand production

One approach to the synthesis of nucleic acids is the single production sequence, single promoter sequence approach. This technique utilizes a single promoter such as T7, or SP6 phage polymerase, or U6 mammalian promoters to drive the production of the production sequence downstream of the promoter. The benefit of this method is the size efficiency of the construct (i.e. promoter sequence and production sequence). The drawback to this method is the inability to concurrently produce multiple strands at the same quantity, efficiency, and in the same compartment (e.g. tube, cell). These methods and compositions (i.e. opposed template) presented in this patent allows the production of two production sequences concurrently, with the same quantity of each production strand produced, with the same efficiency, and within the same compartment. Thus these methods and compositions represent improvements over classical methods of single production sequence, single promoter sequence approaches of nucleic acid strand production. Moreover our experimental evidence suggest that during the production of two complementary strands of DNA or RNA using the opposed template methods and compositions, the complementary strands will anneal to each other thus forming double stranded structures without further processing. Thus these methods and compositions are ideal for the production of large quantities of double stranded RNA or DNA without separate steps for both the sense and antisence strand production processes. Moreover since the opposed template produced strands bind to each other while being produced these methods and compositions also allow for increased efficiency by allowing the user to omit classical annealing steps that are typically necessary to anneal separately produced sense and antisence nucleic acid strands. Additionally, because two different nucleic acid strands can be produced this allows the production of molecules such as ribozymes or deoxyribozymes that can act on the product of the other production template or on a separate nucleic acid strand to allow for complex modifications to either product strand or other molecular target (e.g. cellular mRNA) or allow a layer of regulation or modulation to be added to the production of the nucleic acids. Also because there are two production sequences RNA/DNA hybrid opposed template molecules allow for RNA and DNA to be synthesized at the same time, with the same efficiency and quantity if expressed under similar promoters utilizing equivalent activities of polymerase enzymes. Moreover the design of the opposed template molecule allows for it easy incorporation into other vectors (e.g. plasmids, cosmids, bacteriophages, viruses, extrachromosomal arrays, artificial chromosomes) either for its production by the vector, or for its integration and use within the vector for the production of nucleic acid strands (i.e. RNA, DNA, ribozymes, deoxyribozymes).

Plasmid, cosmid, bacteriophage or viral approaches to the production of nucleic acid strands.

Other methods for the production of RNA or DNA utilize large circular fragments of DNA such as plasmid, cosmids, bacteriophages, or viruses. Plasmids are typically circular double stranded DNA molecules that can contain numerous production sequences. Cosmids are a type of plasmid constructed by the insertion of cos sequences enabling them to be packaged into λ phage particles in vitro. The advantages of plasmids and cosmids include the ability to construct multiple expression regions capable of producing various production sequence products concurrently. The disadvantages of plasmids and cosmids include size, complexity of production, and inefficiency in modification. Plasmids, cosmids and other vectors often require the use of endonucleases in order to splice in production sequences of choice. The opposed template design and method allows for smaller number of nucleotides to be used in the construction of the molecule and thus allows for more efficient production, modification, and also allows for more efficient transfection efficiencies. Moreover, due to its small size the opposed template can be integrate as mentioned above into other vectors for delivery or regulation. Viral and bacteriophage vectors have similar advantages and disadvantages as the aforementioned plasmids and cosmids, however their ability to effect cellular delivery of nucleic acids makes these vectors extremely attractive to genetic engineers. Again, the opposed template design and method allows for smaller number of nucleotides to be used in the construction of the molecule and thus allows for more efficient production, modification, and also allows for safer use versus many viruses and bacteriophages.

### SUMMARY OF THE INVENTION

Methods for the production of duplexes and single-stranded RNA and/or DNA of a desired length and sequence based on a novel template design which incorporates 2 polymerase promoters, primers, and production sequences opposed within a single hybridized molecule are provided. This opposed design allows high-efficiency, high-yield production of single or multiple nucleic acid molecules in a single reaction vessel and thus is amenable to high-throughput automation. This single molecule design also allows easy incorporation of opposed templates into delivery vectors for either in vitro, in vivo, ex vivo, or therapeutic applications. Methods for producing opposed template-based RNA or DNA molecules, or hybrid molecules in vivo and therapeutic uses for such molecules are provided. Opposed template kit designs are also described.

### DRAWING FIGURES

FIG. 1 illustrates the basic design of an opposed template molecule.

FIG. 1A illustrates the basic design of an opposed template molecule

FIG. 2 is a flowchart describing an example of the production and use of an opposed template molecule.

FIG. 3 are digital microscopic pictures of human aortic endothelial cells transfected with opposed template produced small interfering RNA targeting green fluorescent protein, or a non-targeting control. This figure illustrates the ability of the opposed template molecule to produce functional nucleic acid strands for use in techniques such as RNA interference.

### REFERENCE NUMERALS IN DRAWINGS

6 First primary single stranded molecule

8 Second primary single stranded molecule

10 Production sequence of first primary single stranded molecule

12 Promoter complement sequence of first primary single stranded molecule

14 Spacer sequence of first primary single stranded molecule

16 Promoter sequence of first primary single stranded molecule

18 Production sequence of second primary single stranded molecule

20 Promoter complement sequence of second primary single stranded molecule

22 Spacer sequence of second primary single stranded molecule

24 Promoter sequence of second primary single stranded molecule

### DETAILED DESCRIPTION OF THE INVENTION

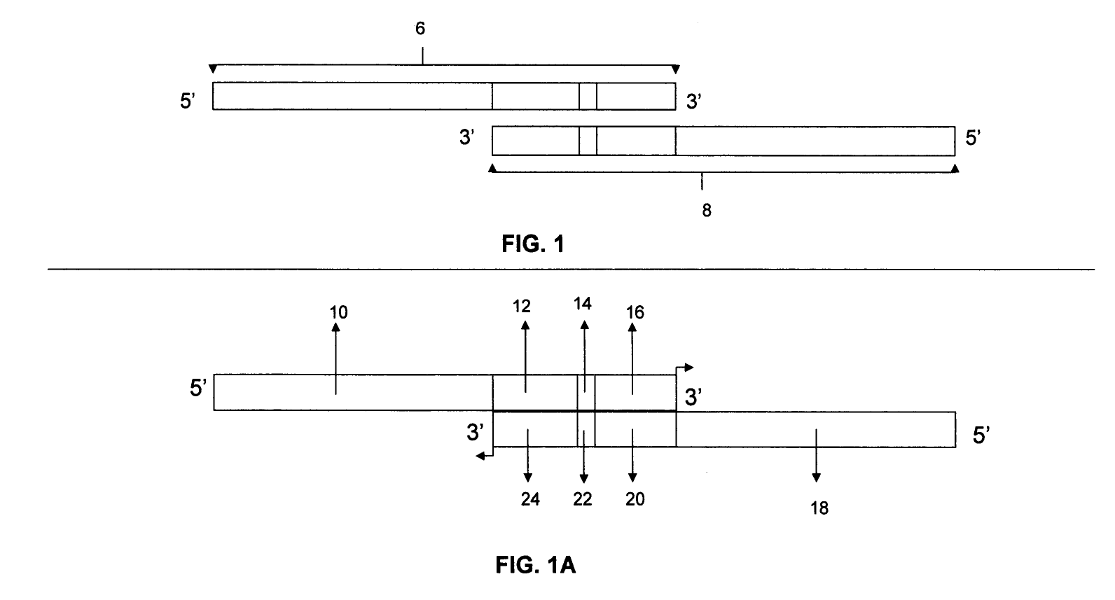

The present invention provides methods for the synthesis of RNA, DNA, or RNA/DNA hybrid molecules via an opposed template single molecule (FIG. 1, FIG. 1A). The molecule is comprised of two primary nucleic acid molecules 6,8 each containing a spacer sequence 14,22, promoter complement sequence 12,20, promoter sequence 16,24, production sequence 10,18. The primary nucleic acid molecules 6,8 are then annealed to form a functional partial double, partial single stranded molecule (i.e. opposed template molecule, FIG. 1). This novel molecule can be utilized to produce RNA, DNA, or RNA/DNA hybrid molecules of desired length and sequence in a single reaction vessel, in vitro, ex vivo, or in vivo.

The term “spacer sequence” 14 refers to any number of nucleotides in a sequence on the first primary nucleic acid molecule 6 that is complementary to the “spacer sequence” 22 on the second primary nucleic acid molecule 8. This sequence is interspersed between the two complete opposed promoter sequences 1 6,20 and 12,24 to allow for efficient polymerase enzyme activity or other functions (e.g. co-activation).

The term “promoter complement sequence” 12 refers to any number of nucleotides in a sequence on the first primary nucleic acid molecule that is complementary to the “promoter sequence” 24 on the second nucleic acid molecule. This sequence is provided to form the “complete promoter” 12,24 and allows for the double stranded promoter of the opposed template. This also applies for the second primary nucleic acid molecule in relation to the first primary nucleic acid molecule.

The term “promoter sequence” 16,24 refers to any number of nucleotides in a sequence that allows for the binding a polymerase and the synthesis of the products from the “production sequence” 10,18.

The term “production sequence” 10,18 refers to any number of nucleotides in a sequence that acts as a template for the partial or complete synthesis of the DNA, RNA, or hybrid DNA/RNA products; that can be either single stranded or duplexes (i.e. products).

In one embodiment the method comprises a production sequence which contains a sequence of any length enabling the production of complementary product that is complementary to itself and contains a “loop sequence” that facilitates the folding and self annealing and duplex formation thus forming double stranded DNA, or RNA, wherein each opposed promoter drives the production of the same or distinct double stranded DNA, or RNA molecule.

The term “loop sequence” refers to a sequence of any number of nucleotides which is non-complementary to itself which allows for the formation of a duplex upstream and downstream from the “loop sequence.”

The opposed template molecule or its products can be delivered in vitro, ex vivo, or in vivo, by incubation, direct injection, transfection, electroporation, transdermally, or orally.

The opposed template molecule can be delivered by the above mentioned methods for synthesis in vitro (i.e. cells), ex vivo, or in vivo

The opposed template molecule can synthesized in vitro (i.e. cells), ex vivo, or in vivo, by incorporation of opposed template molecule into a host genome or by integration of a gene that when transcribed by an endogenous or exogenous polymerase would produce an opposed template molecule in vitro (i.e. cells), ex vivo, or in vivo.

The product can be synthesized by incorporation of opposed template molecule into a delivery vector. A delivery vector can be nothing, virus, bacteriophage, plasmid, liposome, exogenously delivered cells, re-engineered host cell, artificial chromosome, extrachromosomal array, carrier protein, bacteria, fungus, protozoa, plant cell, or other organism.

The product can also be synthesized by the inclusion of the necessary polymerizing enzymes exogenously, and/or endogenously produced via the delivery vector containing both the opposed template and the production sequence for the polymerizing enzyme, or the necessary enzymes can be provided by a separate vector.

The production sequence can code for sequences that allow for integration into vectors, artificial chromosomes, or host genome.

In one of the preferred embodiment of the invention the production sequences are such that they are complementary to each other and can be used for gene silencing via RNA interference.

The present invention may entail the use of the products of the production sequence to silence genes that may be responsible for the maintenance of a cancerous state. A gene that is over expressed in cancerous cells or an inappropriately expressed cancer linked gene may be targeted to inhibit the cancerous growth.

The present invention as disclosed herein may involve the introduction of the opposed template into host cells to render the host cell less susceptible to infection. Such methods may include targeting a gene or set of genes that are necessary for the infecting agent's survival or replication within the host cell.

The present invention can also be used to temporally silence a gene of interest to assay for its function at a certain developmental stage or age of the cell or organism. Since genes are regulated both temporally and spatially the delineation of their role in a temporal fashion would be used to assay for the function of a gene temporally.

In another such embodiment of the present invention, the production sequence products of both strands of the opposed template may remain single stranded and act as antisense mediated silencing agents.

The products of the production sequences can be used to silence a variety of genes of many origins including viral, bacterial, fungal, plant, protozoa, yeast, insect, animal, or mammalian cell genes.

Another preferred embodiment is the production of kits for the purpose of gene silencing via RNA, DNA or both, as well as the production of nucleic acid molecules of a specific sequence for any use (e.g. ribozymes and deoxyribozymes). A kit would include all of the necessary reagents for transcription of nucleic acids and would be performed in a single transcription vessel. This mix would include opposed template molecule(s) of a desired length and sequence as describe above, the corresponding enzyme(s) that would act at the promoter(s) contained within the opposed molecule(s) (e.g. T7 polymerase, U6 polymerase) buffer (e.g. Tris-HCl at pH 8.0, and EDTA) and enzyme transcription buffer (e.g. Tris-HCl at pH 7.9, MgCl2, DTT, NaCl and spermidine), nucleic acid tri-phosphates (NTPs), pyrophosphatase, and RNAase inhibitor, DNAase inhibitor, or both. The mix would then be incubated at the temperature appropriate for polymerization (e.g. 37.5° C. for 2 h). Nucleic acid sequences generated (i.e. products) can then be annealed after stopping the reaction by heating to a high temperature (e.g. 95° C. for 5 min) followed by an annealing temperature (e.g. 1 h at 37.5° C.) to obtain the crude products (e.g. small interfering double-stranded RNA). The mixture can then be further purified by nucleic acid precipitation (e.g. sodium acetate solution at pH 5.2, and then ethanol, dried and resuspended in water). Then the nucleic acid products can be further purified with enzymes (e.g. RNAse A) and gel extraction methods. The final products can then be used for various procedures (e.g. gene silencing, genetic screening, transfection of any cell type, gene therapy).

The present invention may be used for the production of kits for the purpose of gene silencing via RNA, DNA or both, as well as the production of nucleic acid molecules of a specific sequence for any use (e.g. ribozymes and deoxyribozymes). A kit would include all of the necessary reagents for transcription of nucleic acids intracellularly, utilizing host cell enzymes and reagents for the production of nucleic acid molecules of a specific length and sequence. This mix would include opposed template molecule(s) of a desired length and sequence as describe above, the corresponding enzyme(s) (if not provided by the host cell). Next the mix would then be processed to allow proper conditions for incubation/injection with the host cells/organisms (e.g. resuspended in normal saline, or with liposomal transfection reagents) and allowed to induce intracellular production of nucleic acid sequences (i.e. products) which can then be used for various procedures (e.g. gene silencing, gene screening, transfection of any cell type, gene therapy).

As disclosed herein, the production sequence can also code for nucleotide enzymes such as deoxyribozymes and ribozymes that can modify RNA to perform a variety of functions including gene silencing.

The production sequence can produce deoxyribozymes, and ribozymes which can act as RNA replicases and produce double stranded RNA for initiation of RNA interference or other functions. Accordingly the produced ribozymes and deoxyribozymes can be used in any cell type in any organism.

The present invention may entail the use of the opposed template molecule to produce single stranded primers for use by exogenous or endogenous provided enzymes. These primers, or any other types of oligonucleotides for use by exogenous and/or endogenous enzymes can be produced by the opposed template molecule in any organism in vitro, ex vivo, or in vivo.

As disclosed herein the present invention may include methods wherein each of two production sequences of the opposed template molecule codes functionally distinct products, for example, one of the production sequences encodes a deoxyribozyme or ribozyme and the other codes for a hairpin molecule these molecules can be designed to interact with themselves or endogenous or exogenous enzymes or other molecules. Moreover a likely modification of this embodiment can include a method wherein one or both of the produced ribozymes or deoxyribozymes target its opposed template of origin or another opposed template or products of the opposed template origin or other opposed templates.

Another preferred embodiment of the present invention is a method wherein the production sequences are used to induce translational suppression of protein synthesis by encoding products such as microRNAs and other interfering RNAs or DNAs.

Yet another preferred embodiment of the present invention is one where the opposed template molecule, and/or its produced sequences, and/or the necessary reagents, and/or the necessary enzymes corresponding to the promoters/primers on the opposed template molecule, and/or other necessary reagents and molecules needed for production of the products of the production sequence can be delivered via the skin, blood, gastrointestinal tract, eye drops, mucous membrane transfer gels, inhalants, intramuscular injections, intra-tissue implants, tissue/blood grafts, subcutaneous injections, as a contact dust, as a contact liquid, in aerosol form.

Another preferred embodiment is one where the opposed template molecule, and/or its produced sequences, and/or the necessary reagents, and/or the necessary enzymes corresponding to the promoters/primers on the opposed template molecule, and/or other necessary reagents and molecules needed for production of the products of the production sequence can be delivered via the skin, blood, gastrointestinal tract, eye drops, mucous membrane transfer gels, inhalants, intramuscular injections, intra-tissue implants, tissue/blood grafts, subcutaneous injections, as a contact dust, as a contact liquid, in aerosol form and used as antimicrobial/antiviral agents by targeting essential genes of bacterial, fungal, yeast, amoeba, plant, protozoan, insect, mammalian, or animal cells and/or viruses, for gene silencing/interference by the products.

The present invention may entail a method wherein the opposed template molecule, and/or its produced sequences, and/or the necessary reagents, and/or the necessary enzymes corresponding to the promoters/primers on the opposed template molecule, and/or other necessary reagents and molecules needed for production of the products of the production sequence can be delivered via the skin, blood, gastrointestinal tract, eye drops, mucous membrane transfer gels, inhalants, intramuscular injections, intra-tissue implants, tissue/ blood grafts, subcutaneous injections, as a contact dust, as a contact liquid, in aerosol form and used as anti-cancer cell agents by targeting essential cancer cell genes for gene silencing/interference by the products.

In still another preferred embodiment opposed template molecules or products can be utilized for high-throughput genetic screening assaying for gene function, target validation, biological pathway delineations or search for a desired phenotype.

The following examples are meant to be illustrative of the present invention; however, the practice of the invention is not limited or restricted in any way by them.

Opposed Template Directed T7 Synthesis of Small Interfering RNA

The following opposed template, designed to produce small interfering RNA that would silence green fluorescent protein (1), was obtained in desalted DNA oligonucleotide form. Strand-A, 5′-ATG AAC TTC AGG CTC CGA GTT CTA TAG TGA GTC GTA TTA TAA TAC GAC ACT CTA CAT-3′, and Strand-B 5′-CGG CAA GCT GAC CCT GAA GTT CTA TAG TGA GTC GTA TTA TAA TAC GAC TCA CTA TAG-3′. The opposed template molecule was formed by added equal quantities of molecules of Strand-A and Strand-B in a salt buffer designed to promote annealing (e.g. 11 mM Tris-HCl pH 7.9) and the mixture was heated to 95° C. for 5 minutes and allowed to cool to 37° C. the for 2 hours then allowed to cool to room temperature for another 2 hours. Opposed template directed T7 driven transcription was performed similarly to previously described reports (2). Briefly, transcription buffer consisted of the following: 42 mM Tris-HCl pH 7.9, 11 mM NaCl, 4.5 mM MgCl2, 2.5 mM spermidine, and 11 mM DTT. To this was added: 0.15 units yeast pyrophosphatase, 2 mM rNTP, 40 units RNase inhibiting peptide and 100 units T7 RNA polymerase. Also added was 100 pmol annealed opposed template molecule. Next mix was incubated at 37° C. for 1 hour and 30 minutes, after which 1 unit of DNAse-I was added and then incubated for 30 min to remove used opposed template. Generated RNA formed sense and antisense strands of a small interfering RNA duplex designed to silence green fluorescent protein. The mixture was partially purified by precipitation by addition of ice cold 0.1 volumes of 3M sodium acetate (pH 5.1), and 1 volume of isopropanol and allowed to incubate for 10 minutes on ice. Next mix was centrifuged at −20° C. at max speed (10 Kxg) for 30 minutes. The pellet was washed twice with 75% ethanol and dried, and then resuspended in pure water by heating at 55° C. for 10 minutes. This RNA was then frozen until use at −80° C.

Analysis of Opposed Template Produced RNA

Opposed template produced RNA was analyzed by ethidium bromide agarose gel electrophoresis in comparison with 50 base pair DNA marker. As expected the produced RNA was of expected size, separating similarly in the agarose gel as the 50 base pair marker. To further analyze this RNA, human aortic endothelial cells were co-transfected with a plasmid expressing green fluorescent protein and small interfering RNA produced by the opposed template molecule designed to silence GFP or with a control non-targeting small interfering RNA. Results showed that GFP was silenced by microscopic analysis of GFP expression by plasmid transfected human aortic endothelial cells (FIG. 3). Results also showed that control non-targeting small interfering RNA produced undetectable silencing of GFP.

### REFERENCES

1. Caplen, N. J., Parrish, S., Imani, F., Fire, A. and Morgan, R. A. (2001) Specific inhibition of gene expression by small double-stranded RNAs in invertebrate and vertebrate systems. Proc. Natl Acad. Sci. USA, 98, 9742-9747.

2. Milligan, J. F. and Uhlenbeck, O. C. (1989) Synthesis of small RNAs using T7 RNA polymerase. Methods Enzymol., 180, 51-62.

What is claimed is:

1. A method of producing RNA, DNA, or hybrid RNA/DNA molecules having a defined length and sequence comprising: providing first 2 primary single-stranded nucleic acid molecules containing a variable length spacer sequence, promoter complement, promoter, and production sequences. The 2 primary single-stranded molecules can be of heterogeneous or homogeneous sequence, wherein the promoter complement sequence is complementary to the promoter of the second single-stranded nucleic acid molecule, and wherein the second single-stranded nucleic acid molecule contains a promoter complement sequence that is complementary to the promoter sequence of the first strand. These nucleic acid molecules are then annealed into one molecule to form a partial duplex, partial single stranded nucleic acid template, where the promoter sequences are aligned in opposing directions, wherein an endogenous or exogenously provided polymerase is used to drive the production of RNA, DNA, or RNA/DNA hybrids, in vitro, ex vivo, or in vivo.

2. A method according to claim 1 wherein the spacer sequence can be zero to any number of base pairs, wherein the spacer sequence of both primary single-stranded molecules are complementary to each other or aid the formation of a hairpin loop. The spacer sequence can be a functional promoter element, promoter modifying element, or a nucleic acid sequence that simply links (i.e. linker sequence) other nucleic acid sequences together.

3. A method according to claim 1 wherein the promoter complement consists any sequence on the first primary strand that is complementary to the any promoter sequence on the second opposing primary strand, and wherein the promoter complement on the second primary strand is complementary to any promoter sequence on the first opposing primary strand. This complementary sequence may or may not be a functional promoter element or a promoter modifying element.

4. A method according to claim 1 wherein the promoter consists of any sequence modulating the binding and subsequent initiation of polymerization of the product as read from the product sequence by any polymerizing enzyme, or modifiers of polymerizing enzymes.

5. A method according to claim 1 wherein the production sequence contains any sequence of any length enabling the production of an RNA, DNA, or RNA/DNA hybrid product.

6. A method according to claim 1 wherein the opposed template molecule or its products is delivered in vitro, ex vivo, or in vivo, by direct injection, transfection, electroporation, transdermally, orally, or liquid.

7. A method according to claim 1 wherein the production sequence codes for a self-annealing RNA duplex having a defined length and sequence comprising: providing a primary single-stranded RNA to generate an RNA of defined length and sequence which is self-complementary over at least a portion of its length, and self-annealing thus forming a hairpin RNA duplex.

8. A method according to claim 1 wherein products are synthesized by in vitro or in vivo transcription.

9. A method according to claim 1 wherein products are synthesized by incorporation of opposed template molecule the host genome or into a delivery vector wherein a delivery vector can be a virus, bacteriophage, plasmid, liposome, exogenous cell, re-engineered host cell, artificial chromosome, extrachromosomal array, carrier protein, carrier compound, or artificial chromosome.

10. A method according to claim 1 wherein the polymerizing enzymes necessary for opposed template product production are vector delivered with the opposed template molecules or contained within the host organism or genome, provided by organism associated flora, or provided upon host infection by virus or organism.

11. A method according to claim 1 wherein the production sequences produce complementary products of RNA, DNA or both RNA and DNA that will be used to form duplexes of any length that can be used to sequence specifically silence genes.

12. A method according to claim 7 wherein the opposed template molecule produced self-complementary RNA or DNA duplexes can be used for gene silencing.

13. A method according to claim 1 wherein the production sequence products of both strands of the opposed template may remain single stranded.

14. A method according to claim 1 wherein the opposed temple molecule formed single stranded products can be used for gene silencing.

15. A method according to claim 11 wherein the target gene to be silenced is that of any cell type (e.g. bacterial, fungal, plant, protozoan, animal, insect, mammalian), or any virus type.

16. A method according to claim 1 wherein the production sequence codes for ribozymes or deoxyribozymes.

17. A method according to claim 16 wherein the produced ribozymes and deoxyribozymes can be used in any cell type in any organism.

18. A method according to claim 1 wherein the opposed template molecule, and/or its produced sequences, and/or the necessary reagents, and/or the necessary enzymes corresponding to the promoters/primers on the opposed template molecule, and/or other necessary reagents and molecules needed for production of the products of the production sequence can be delivered via the skin, blood, gastrointestinal tract, eye drops, mucous membrane transfer gels, inhalants, intramuscular injections, intra-tissue implants, tissue/blood grafts, subcutaneous injections, as a contact dust, as a contact liquid, in aerosol form, via stem cells, via genetically engineered cancer cells, via genetically engineered patient-harvested cells, via genetically engineered normal cells, via genetically engineered bacteria, via genetically engineered viruses, via genetically engineer fungi, via genetically engineered protozoa, via genetically engineered plants or via genetically engineer bacteriophages, via carrier proteins, or carrier compounds, and used as a treatment for a disease or condition.

19. A method according to claim 1 wherein opposed template molecules can be utilized for high-throughput genetic screening assaying for gene function, protein expression and/or phenotype.

20. A method according the claim 1 wherein a kit can be designed for various uses (e.g. small interfering RNA synthesis, genetic screening, oligonucleotide synthesis, antisense gene silencing, microRNA synthesis for translational interference) wherein the opposed template molecule is a component.
